(19) United States
(12) Patent Application Publication
(10) Pub. No.: US 2004/0152987 A1
(43) Pub. Date: Aug. 5, 2004

(54) Inspection system and inspection method

(75) Inventor: Michael Haisch, Aalen (DE)

Correspondence Address: Steven M. duBois, Potomac Patent Group, PLLC, PO Box 855, McLean, VA, 22101-0855, US

(73) Assignee: Carl-Zeiss-Stiftung trading as Carl Zeiss, Heidenheim (DE)

(21) Appl. No.: 10/703,561

(22) Filed: Nov. 10, 2003

(30) Foreign Application Priority Data
Nov. 11, 2002 (DE) 102 52 313.4

(51) Int. Cl. A61B 6/00

(52) U.S. Cl. 600/473; 600/476

(57) ABSTRACT

An inspection system and an inspection method are provided for visualizing a fluorescent marker and a tissue region surrounding the fluorescent marker, the method comprising:

illuminating the tissue region with light of a first wavelength range comprising at least a portion of an excitation spectrum of the fluorescent marker, wherein the fluorescent marker, upon excitation, emits light of a fluorescence spectrum;

illuminating the tissue region with light of at least one second wavelength range which is substantially free of light having wavelengths contained in the fluorescence spectrum and which comprises a partial wavelength range which is substantially free of light having wavelengths contained in the excitation spectrum; and

changing an intensity of the light of the first wave length range relative to an intensity of the light of the at least one second wavelength range.

### BACKGROUND OF THE INVENTION

1. Field of the Invention

The present invention relates to an inspection system and a corresponding method for visualization of at least one fluorescent marker simultaneously with a surrounding tissue surrounding the at least one fluorescent marker.

2. Brief Description of Related Art

In a conventional microscopic method for inspecting a tissue region, a tumor tissue contained therein is made visible by supplying a fluorescent marker to the tissue before the inspection; the fluorescent marker accumulates in the tumorous tissue, such that the tumorous tissue is marked by the fluorescent marker. Under irradiation of the tissue region with light, which contains substantially only wavelengths of an excitation spectrum of the fluorescent marker, the fluorescence of the tumor tissue, generated by the fluorescent marker, is perceptible by the eye of an observer, and thus the tumor can be localized within the tissue region.

Herein, the light illuminating the tissue region is restricted to wavelengths in which the fluorescence is excited. Thus the fluorescing tumor, luminescing within an almost dark surroundings, can be identified. On the other hand the observer also wants to analyze the surroundings of the tumor. To this end, an intense white light source is switched on. But this light source is conventionally so bright, that the fluorescence can hardly be perceived. For this reason the surgeon switches the white light source alternately on and off, so that he receives an impression of both the location of the tumor and of the tissue surrounding the same.

This procedure is bothersome and demands highest concentration from the observer, as he has to memorize the image that resulted from the respectively other illumination mode used before.

### SUMMARY OF THE INVENTION

It is an object of the present invention, to provide an inspection system, which allows for an improved observation of a fluorescent marker within a non-fluorescing tissue region surrounding the marker. It is also an object of the present invention to provide a corresponding method.

This object is solved by an inspection system for visualization of a fluorescent marker and a tissue region surrounding the same, wherein the inspection system includes an illumination system for irradiation of the tissue region with a suitable light. The illumination system includes an excitation light source arrangement for providing light in a first wave length range comprising at least a portion of an excitation spectrum of the fluorescence marker, wherein the fluorescence marker, upon excitation, emits light of a fluorescence spectrum. In the context of the present application the term “excitation spectrum” designates a range of wave lengths, in which the fluorescence shows an excitation efficiency which is higher than 10%, and in particular higher than 20%, of a maximum excitation efficiency.

When excited with light of the excitation spectrum, the fluorescent marker emits light of a fluorescence spectrum. In the context of this application the term “fluorescence spectrum” designates wavelengths of a range in which, at a predefined excitation, the intensity of the generated fluorescence radiation is higher than 10%, and in particular higher than 20%, of a maximum fluorescent intensity in this range.

The illumination system further includes an illumination light source arrangement for providing light of at least one second wavelength range of the visible light which is substantially free of light having wavelengths contained in the fluorescence spectrum and which includes a partial wavelength range which is substantially free of light having wavelengths contained in the excitation spectrum. The light, provided by the illumination light source arrangement, is used to illuminate the surroundings of the marker such that the observer can perceive the surroundings, whereas the excitation light source arrangement is used for exciting the fluorescence of the fluorescent marker.

The illumination system is configured such that an intensity of the illumination light source arrangement may be changed relative to an intensity of the excitation light source arrangement. It is thus possible to adjust a brightness of the visible light reflected by the surroundings of the fluorescent marker relative to an intensity of the fluorescence such that both the fluorescent marker and the tissue region surrounding the fluorescent marker are perceivable simultaneously with sufficient contrast, and such that the light reflected from the tissue region does not outshine the fluorescence.

According to a preferred embodiment, the second wave length range is free of wavelengths contained in the excitation wavelength range. Therewith the fluorescent marker will not be excited when the excitation light source arrangement is switched off and merely the illumination light source arrangement is in operation. Since the fluorescent marker is not excited, a substance providing the fluorescent marker will not be used up or will not bleach. Further, an amount of fluorescence excitation will not change if the intensity of the light provided by the illumination light source arrangement changes the amount of fluorescence excitation may be remained at a maximum amount, accordingly.

According to a further preferred embodiment, the first wavelength range and the second wavelength range are substantially non-overlapping wavelength ranges such that the illumination and the excitation of the fluorescence may be adjusted independently from each other.

Preferably the second wavelength range is arranged in between the first wavelength range and the wavelengths contained in the fluorescence spectrum. For example, the first wavelength range with the excitation spectrum includes blue light to ultra-violet light, the second wavelength range includes green light, and the fluorescence spectrum includes red light. It is then possible that the fluorescence spectrum stimulates red uvulas in a retina of an eye of the observer and that the illumination light stimulates the green uvulas of the observer. Due to the excitation of different uvulas of the eye the fluorescent marker may be perceived with a high contrast and well-differentiated to the surrounding tissue.

According to a further preferred embodiment, the excitation light source arrangement and the illumination light source arrangement each have a separate light source for emission of the light in the first and the second wavelength ranges, respectively. Due to the provision of the independent light sources for the generation of the light of both wavelength ranges, the intensities thereof may be easily adjusted relative to each other.

However, according to a further preferred embodiment, the excitation light source arrangement and the illumination light source arrangement comprise a common broad-band light source for emitting light of both the first wavelength range and the second wavelength range.

According to a further preferred embodiment, an illumination light filter is provided for adjusting the intensities of the light emitted in the first and second wavelength ranges relative to each other, wherein a transmission of the illumination light filter in the second wavelength range is adjustable. By changing the transmission of the illuminating light filter, the intensity of the light in the second partial wavelength range can be changed relative to the intensity of the first partial wavelength range, wherein the latter intensity remains substantially unchanged.

According to a further preferred embodiment, the illumination light filter is provided by a rotatable filter disc.

According to a further preferred embodiment, the illumination light filter is provided by a controllable liquid-crystal filter such that the transmission of the illumination light filter is changeable without any mechanical movement of components.

According to a further preferred embodiment, a fluorescence color filter is provided in the illumination system to make the fluorescence perceivable with an optimal contrast, wherein the fluorescence color filter substantially does not transmit light within the fluorescence spectrum.

According to a further preferred embodiment, the intensities of the first wavelength range and the second wavelength range may be automatically adjusted relative to each other based on a signal generated by a camera provided for obtaining an image of the tissue region. A controller is provided for analyzing the image obtained by the camera with respect to light intensities of the obtained image in the excitation spectrum and the second wavelength range. Based on the analyzed light intensities, the controller may change the relative intensities of light provided by the illumination system such that the light intensities in the image have a desired relation to each other.

### BRIEF DESCRIPTION OF THE DRAWINGS

The forgoing as well as other advantageous features of the invention will be more apparent from the following detailed description of exemplary embodiments of the invention with reference to the accompanying drawings, wherein:

FIG. 1 illustrates a first embodiment of an inspection system in accordance with the present invention;

FIG. 2 shows a representation of an excitation spectrum and a fluorescence spectrum of a fluorescent marker which may be used in the inspection system of FIG. 1;

FIG. 3 is an illustration of transmissions of a color filter used in the inspection system of FIG. 1;

FIG. 4 is an illustration of transmissions of a color filter used in the inspection system of FIG. 1; and

FIG. 5 illustrates an inspection system according to a second embodiment of the present invention.

### DETAILED DESCRIPTION OF EXEMPLARY EMBODIMENTS

In the embodiments described below, components with are identical in function and structure are designated as far as possible by the same reference numerals. Therefore, to understand the features of the individual components of a specific embodiment, the descriptions of other embodiments should be referred to.

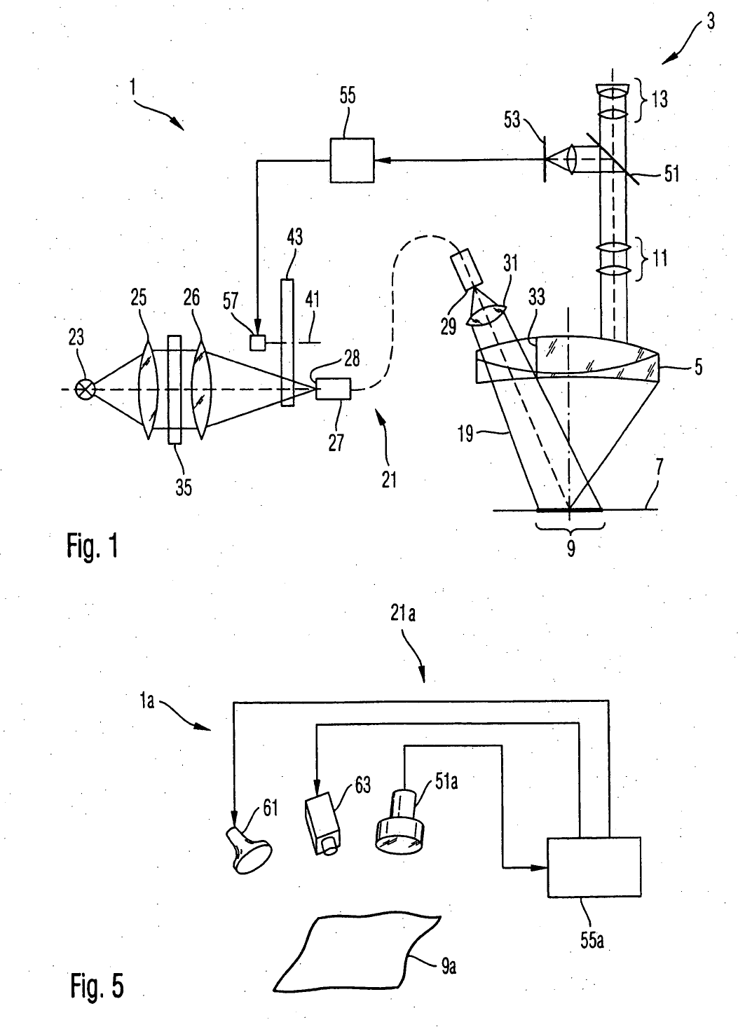

An inspection system 1 schematically shown in FIG. 1 comprises a stereo microscope 3 including an objective lens 5 having an object plane 7 in which a tissue region 9 to be inspected is arranged. The stereo microscope 3 has two optical beam paths arranged spaced-apart from each other. Each beam path includes a zoom system 11 and an eyepiece 13. Only one of the two optical beam paths is shown in FIG. 1. Looking into the eye-pieces 13, the user may observe a magnified stereoscopic representation of the tissue region 9. For the purpose of the present example, the tissue region 9 contains a tumor which is enriched with a fluorescent marker.

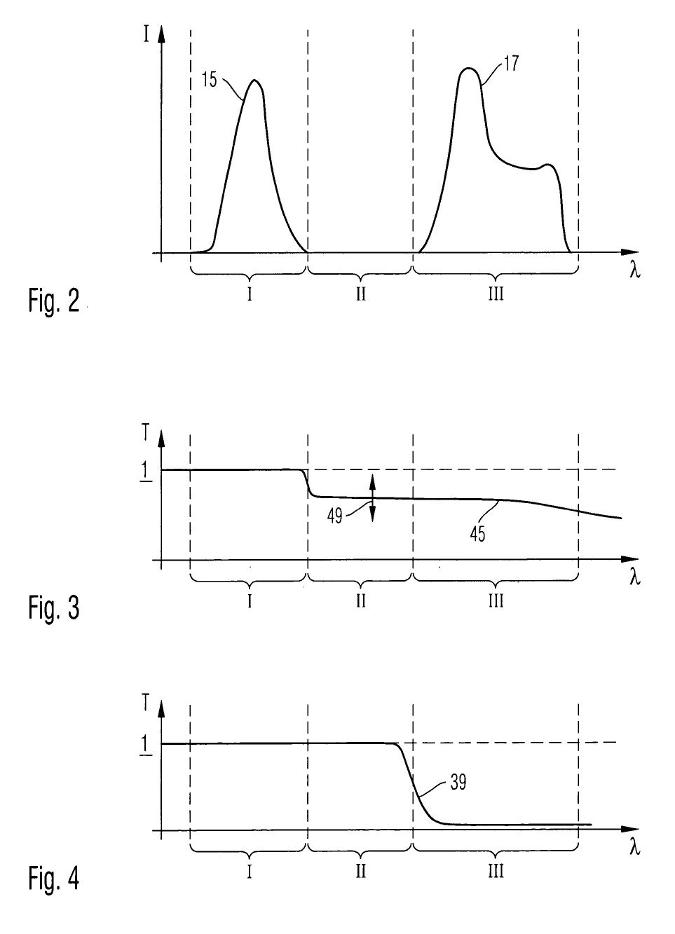

FIG. 2 shows an excitation spectrum 15 and a fluorescence spectrum 17 of the fluorescent marker in dependence of the wavelength λ and in arbitrary units of the intensity I. Further, FIG. 2 indicates three wavelength ranges, namely a wavelength range I from 400 nm to 500 nm, a wavelength range II from 500 nm to 600 nm and a wavelength range III from 600 nm to 700 nm.

A dye used as the fluorescent marker is Protoporphyrin IX. In the human body Protoporphyrin IX is formed from a precursor, which is called 5-Aminolevulinic Acid, which may be obtained from the company “Medac Gesellschaft für klinische Spezialpräparate mbH”, Hamburg, Germany.

The excitation spectrum is substantially completely contained in the wavelength range I, the fluorescence spectrum 17 is substantially completely contained in the wavelength range III, and the wavelength range II is arranged between the two wavelength ranges I and III.

An illumination system 21 is provided for illuminating the tissue region 9 with an appropriate light beam 19. The illumination system comprises a broad-band light source 23, for example a Halogen lamp. Light emitted from the light source 23 is collimated by a collimation system having two lenses 25, 26 such that the light is injected with a high efficiency into an input end 28 of a glass fiber bundle 27. The light injected into the glass fiber bundle 27 is emitted from an emitting end 29 of the glass fiber bundle 27, collimated by a collimator 31, and traverses the objective lens 5 through a cut-out aperture 33 provided in the objective lens 5.

Between the collimating lenses 25 and 26 there is arranged a fluorescence color filter 35. A transmission T of the fluorescence color filter 35 in dependence of the wavelength λ is shown as a curve 39 in FIG. 4. In the wavelength ranges I and II the fluorescence color filter is substantially completely transparent, and in the wavelength range III it is substantially non-transparent. Herewith it is substantially excluded that the illumination system 21 illuminates light of the wavelength range III, i.e. light of the fluorescence spectrum 17, onto the tissue region 9. Thus, at least the surroundings of the tumor, in which substantially no fluorescent marker is accumulated, does substantially not reflect radiation in the range of the fluorescence spectrum.

A rotatable filter disc 41 carrying an illumination light filter 43 is arranged between the input end 28 and collimating lens 26. A transmission T of the illumination light filter 43 in dependence of the wavelength λ is shown in FIG. 3 as a curve 45. The illumination light filter is substantially completely transmitting in the wavelength range I such that the radiation provided by the broad-band light source 23 is incident with a high intensity on the tissue region 9 in order to excite the fluorescence of the fluorescent marker in that region.

The transmission of the illumination light filter 43 is smaller than unity in the wavelength range II. The transmission of the illumination light filter 43 is also smaller than unity in the wavelength range III, wherein the dependency of the transmission of the illumination light filter 43 is in this wavelength range III is without relevance, as light transmitted by the illumination light filter 43 is suppressed in the optical beam path of the illumination system 21 anyway.

The rotatable filter disc 43 is configured such that regions of a continuously decreasing transmission in the wavelength range II are arranged along a circumferential direction of a periphery thereof. Thus, by changing an angular orientation of the rotatable filter disc 41, an adjustment of the light intensity in the wavelength range II coupled into the glass fiber bundle 27 is possible as it is indicated with an arrow 49 in FIG. 3.

By rotating the rotatable filter disc 41 it is possible to change the relative intensities of light in the wavelength ranges I and II of the light beam 19 illuminating the issue region 9.

A semitransparent mirror 51 is arranged between the zoom system 11 and the eye-piece 13 in one of the two stereoscopic beam paths of the microscope 3. The semitransparent mirror feeds some light to the beam path towards a camera 53 such that the camera may detect an image of the object plane 7. The image output from the camera 53 is transmitted to a computer 53 which analyses the image with respect to light intensities within the wavelength ranges II and III. In particular, the computer 55 identifies locations of a maximum intensity within the image for each of the wavelength ranges II and III.

Further, an optimal relation of these maximal intensities is stored in the computer 55. This optimal relation has been determined in advance such that a user, who looks into the eye-piece 13, perceives fluorescing regions and adjacent non-fluorescing regions of the tissue simultaneously with a high contrast, without the fluorescence radiation being outshined by the region of the tissue 9 surrounding the fluorescing region. The computer 55 controls a motor 57 for rotating the rotatable filter disc 41 such that the relation of the maximal intensities in the wavelength range II and the wavelength range III substantially corresponds to the optimal relation.

A schematically illustrated inspection system 1a for observing a tissue region 9a shown in FIG. 5 comprises an illumination system 21a including an illumination light source 61 for generating light having wavelengths which are substantially completely contained in the wavelength range II of FIG. 2, and a fluorescence excitation light source 63, such as a laser or a LED, which emits light of about 450 nm, i.e. in the wavelength range I as shown in FIG. 2. Light beams generated by the fluorescence excitation light source 63 and the illumination light source 61 also are directed onto the tissue region 9a. The wavelength of about 450 nm is chosen to correspond to the maximum of the excitation spectrum 15.

A camera 51a generates an image of the tissue region 9a and transmits corresponding image data to the computer 55a. The computer 55a analyses the image data as illustrated with reference to FIG. 1 above. The computer 55a then changes the intensity of the illumination light source 61 such that an optimal relation between maximal intensities of the wavelength ranges II and III is obtained.

Thus, the user may observe the tissue region 9a with his naked eyes or with a magnifying glass, such as a head-carried magnifying glass, and he can perceive fluorescing regions simultaneously with regions surrounding the fluorescing regions of the tissue region 9a simultaneously and at a high contrast.

As an alternative to using the illumination light filter 43 of the embodiment illustrated with reference to FIG. 1, it is also possible to use a liquid crystal filter having an adjustable transmission in wavelength range II as the illumination light filter 43.

It is further possible that the excitation light source arrangement generates light only in a wavelength range which is narrower than the excitation spectrum of the fluorescent marker. Such narrow-band source may be a laser or a LED.

Summarized, the invention provides an inspection system and an inspection method for visualizing a fluorescent marker and a tissue region surrounding the fluorescent marker. The method comprises illuminating the tissue region with light of a first wavelength range comprising at least a portion of an excitation spectrum of the fluorescent marker, wherein the fluorescent marker, upon excitation, emits light of a fluorescence spectrum; illuminating the tissue region with light of at least one second wavelength range which is substantially free of light having wavelengths contained in the fluorescence spectrum and which comprises a partial wavelength range which is substantially free of light having wavelengths contained in the excitation spectrum; and changing an intensity of the light of the first wave length range relative to an intensity of the light of the at least one second wavelength range.

Therefore, while the present invention has been shown and described herein in what is believed to be the most practical and preferred embodiments, it is recognized that departures can be made therefrom within the scope of the invention, which is therefore not be limited to the details disclosed herein but is to be accorded the full scope of the claims so as to embrace any and all equivalent methods and apparatus.

What is claimed is:

1. An inspection system for visualizing a fluorescent marker and a tissue region surrounding the fluorescent marker, the inspection system comprising:
an illumination system including an excitation light source arrangement for supplying light of a first wave length range to the issue region, the first wavelength range comprising at least a portion of an excitation spectrum of the fluorescent marker, wherein the fluorescent marker, upon excitation, emits light of a fluorescence spectrum,
the illumination system further including an illumination light source arrangement for supplying light of at least one second wavelength range to the issue region, wherein the second wavelength range is substantially free of light having wavelengths contained in the fluorescence spectrum and which comprises a partial wavelength range which is substantially free of light having wavelengths contained in the excitation spectrum,
wherein the illumination system is configured such that an intensity of the light of the first wave length range provided by the excitation light source arrangement is changeable relative to an intensity of the light of the at least one second wavelength range provided by the illumination light source arrangement.

2. The inspection system according to claim 1, wherein the second wavelength range is free of wavelengths contained in the excitation spectrum.

3. The inspection system according to claim 1, wherein the first wavelength range and the second wavelength range are substantially non-overlapping wavelength ranges.

4. The inspection system according to claim 1, wherein the second wavelength range is in between the first wavelength range and the wavelengths contained in the fluorescence spectrum.

5. The inspection system according to claim 1, wherein the excitation light source arrangement comprises an excitation light source for emitting light in the first wavelength range and wherein the illumination light source arrangement comprises an illumination light source separate from the excitation light source, for emitting light in the second wavelength range.

6. The inspection system according to claim 1, wherein the excitation light source arrangement and the illumination light source arrangement have a common light source for emitting light of the first wavelength range and of the second wavelength range.

7. The inspection system according to claim 6, wherein the illumination system further includes an illumination light filter having an adjustable transmission in the second wavelength range.

8. The inspection system according to claim 7, wherein the illumination light filter comprises a rotatable filter disc.

9. The inspection system according to claim 7, wherein the illumination light filter comprises a liquid crystal filter.

10. The inspection system according to claim 1, wherein the illumination light source arrangement further includes a color filter which is substantially non-transparent for light contained in the fluorescence spectrum.

11. The inspection system according to claims 1, further comprising a camera for obtaining an image of the tissue region with light of wavelength ranges which comprise at least the wavelengths of the fluorescence spectrum and at least a portion of the second wavelength range, and further comprising a controller for changing the intensity of the light of the first wave length range provided by the excitation light source arrangement relative to the intensity of the light of the at least one second wavelength range provided by the illumination light source arrangement based on an image intensity at at least one location in the image.

12. The inspection system according to claim 1, further comprising a microscopy system for generating a magnified representation of the tissue region for an observer.

13. A method of visualizing a fluorescent marker and a tissue region surrounding the fluorescent marker, the method comprising:
illuminating the tissue region with light of a first wavelength range comprising at least a portion of an excitation spectrum of the fluorescent marker, wherein the fluorescent marker, upon excitation, emits light of a fluorescence spectrum;
illuminating the tissue region with light of at least one second wavelength range which is substantially free of light having wavelengths contained in the fluorescence spectrum and which comprises a partial wavelength range which is substantially free of light having wavelengths contained in the excitation spectrum; and
changing an intensity of the light of the first wave length range relative to an intensity of the light of the at least one second wavelength range.
